SEQUENCE LISTING (8 sequences)

SEQ ID NO: 1 - protein, 15 aa, Human immunodeficiency virus
GIKQLQARVLAVERY

SEQ ID NO: 2 - protein, 15 aa, Human immunodeficiency virus
NEQLLELDKWASLWN

SEQ ID NO: 3 - protein, 15 aa, Human immunodeficiency virus
EKDLLALDKWANLWN

SEQ ID NO: 4 - protein, 15 aa, Human immunodeficiency virus
NEQDLLALDSWNLWN

SEQ ID NO: 5 - protein, 15 aa, Human immunodeficiency virus
EQELLKLDQWASLWN

SEQ ID NO: 6 - protein, 20 aa, Homo sapiens
FTRSQKEGLHYTCSSHFPYS

SEQ ID NO: 7 - protein, 7 aa, Human immunodeficiency virus
ELDKWAS

SEQ ID NO: 8 - protein, 10 aa, Human immunodeficiency virus
IHIGPGRAFV

(19) United States
(12) Patent Application Publication
(10) Pub. No.: US 2005/0287156 A1
(43) Pub. Date: Dec. 29, 2005

(54) Novel amine-based adjuvant

(75) Inventors: Ulf Schroder, Sundbyberg (SE); Jorma Hinkula, Nacka (SE)

Correspondence Address: BACON & THOMAS, PLLC, 625 SLATERS LANE, FOURTH FLOOR, ALEXANDRIA, VA, 22314, US

(73) Assignee: Eurocine AB, Stockholm (SE)

(21) Appl. No.: 11/137,779

(22) Filed: May 26, 2005

Related U.S. Application Data
(63) Continuation of application No. PCT/EP03/13348, filed on Nov. 26, 2003.

(30) Foreign Application Priority Data
Nov. 26, 2002 (DK) PA 2002 01827

(51) Int. Cl. A61K 39/00; A61K 39/12; A61K 39/04

(52) U.S. Cl. 424/184.1; 424/204.1; 424/234.1

(57) ABSTRACT

An adjuvant for use in a vaccine, the adjuvant comprising one or more cationic substances such as, e.g., acyl amines comprising from 4 to 30 carbon atoms, 5 quaternary ammonium compounds derived from acyl amines, cationic acyl amides, amino acids conjugated to an acyl group, etc., and mixtures thereof.

### FIELD OF THE INVENTION

The present invention relates to a novel adjuvant composition for administration of a polynucleotide or a polypeptide to an animal in order to enhance the immunological response against the polypeptide, or the polypeptide expressed as a result of the administration of the polynucleotide.

### BACKGROUND OF THE INVENTION

Within the last decade plasmids encoding antigens have revolutionized vaccine design. Although no DNA vaccine has yet been approved for routine human or veterinary use, the potential of this vaccine modality has been demonstrated in experimental animal models.

Plasmid DNA vaccination has shown efficacy against viral, bacterial and parasitic infections modulated the effects of autoimmune and allergic diseases and induced control over cancer progression. Because of the simplicity and versatility of these vaccines, various routes and modes of delivery are possible to engage the desired immune responses. These may be T or B effector cell responses able to eliminate infectious agents or transformed cells. DNA vaccines may also induce an immunoregulatory/modulatory or immunosuppressive (tolerizing) response that interferes with the differentiation, expansion or effector functions of B and T cells. Pre-clinical and initial small-scale clinical trials have shown DNA vaccines in either of these modes to be safe and well tolerated. DNA vaccines have proven very effective in small animal models and are also effective in humans and larger animals, including primates, cattle, horses, and swine.

However, when DNA is administered alone, the administration of a relatively high amount of DNA (several milligrams) is required to give a long-lasting and strong immune response in humans and larger animals.

However, although the use of DNA vaccines at milligram doses is feasible, it would impose serious limitations on the number of constructs that could be included in a vaccine. In addition, the use of very high doses of DNA is less favorable from a process economic standpoint. Therefore, there is a clear need to induce effective immunity in humans with lower and fewer doses of DNA, as well as to increase the magnitude of the immune responses obtained.

A number of such systems that can evoke a protective immune response have been tested, and the majority of these rely on the use of cationic molecules that are co-administered with the DNA.

Substances that has been described in the literature includes lactic acid particles with dioleyl-1,3-trimethylammonioproane (PNAS 2000, 98(2) 811), liposomes containing DMRIE/DOPE (Vaccine 1997, 15(8) 818), Vaxfectin liposomes (Vaccine 2001, 19, 1911-23) and poly-l-arginine combined with CpG motifs is described in Vaccine 20 (2002) 3498-3508.

Furthermore, cationic emulsions were made by the inclusion of either 1,2-dioleoyl-3-trimethyl-ammonium-propane (DOTAP) or the Th1 inducing adjuvant dimethyl, dioctadecyl ammonium bromide (DDA) (Vaccine 20 (2002) 3389-3398). A cationic polysaccharide, Chitosan, has also been described as a delivery system for DNA and poly-peptides (Advanced Drug Delivery Reviews 51 (2001) 81-96).

### DETAILED DESCRIPTION OF THE INVENTION

As described above, one of the main problems of DNA-based vaccines is their poor generation of antibodies upon administration to humans. Thus, the present invention provides adjuvants that are able to increase and/or modulate the immune response of an organism to nucleic acid-based or peptide-based vaccines and/or antigens.

Furthermore, as compared to many of the known DNA adjuvants, the adjuvants according to the invention show a very high degree of stability, i.e. it is possible to store them at room temperature for more than 6 months.

The adjuvants according to the invention comprise one or more positively charged (cationic) substances. When the adjuvants are administered together with polynucleotdes, which normally have a negative charge, or polypeptides, which may be either negatively or positively charged, there will be electrostatic interactions between the components. Such interactions are of vital importance in order to obtain an enhanced immune response.

Accordingly, the invention relates to an adjuvant for use in a vaccine, the adjuvant comprising one or more cabonic substances such as, e.g., acyl amines comprising from 4 to 30 carbon atoms, quaternary ammonium compounds derived from acyl amines, cationic acyl amides, amino acids conjugated to an acyl group, etc., and mixtures thereof.

Definitions

Throughout the text including the claims, the following terms shall be defined as indicated below.

The term “acyl” or “acyl group” encompasses natural or synthetic, branched or unbranched, cyclic or acyclic, substituted or unsubstituted acyl, alkyl, alkenyl and alkynyl chains of from 4 to 30 carbon atoms, such as, e.g., from 6 to 24 carbon atoms, from 8 to 20 carbon atoms or from 12 to 20 carbon atoms.

The term “acyl amine” encompasses acyl, alkyl, alkenyl or alkynyl chains onto which an amine group has been attached.

The term “antigen” is defined as anything that can serve as a target for an immune response. The immune response can be either cellular or humoral and be detected in systemic and/or mucosal compartments.

The term “vaccine” is defined herein as a suspension or solution of antigenic moieties, usually consisting of infectious agents, or some part of the infectious agents, that is introduced to an animal body to produce active immunity.

The term “adjuvant” as used herein is any substance whose admixture with an injected immunogen increases or otherwise modifies the immune response.

The term “gp41” means the gp41 transmembrane HIV envelope glycoprotein and any fragments or variants, alleles, analogs and derivatives thereof.

By the term “gp41 nucleotide” is intended to mean a nucleotide encoding the gp41 protein or fragments thereof.

The term “L3” as used herein is defined as an adjuvant for classic vaccines comprising a monoglyceride and a fatty acid as described in PCT/SE97/01003.

The term “N3” as used herein is defined as the adjuvant formulation according to the present invention.

The cationic substances in the adjuvant described above may comprise at least one group comprising a nitrogen atom, such as a primary, secondary or tertiary amine, a quaternary ammonium compound, a primary, secondary or tertiary amide or an amino acid.

The cationic substance may further comprise at least one acyl group, wherein the acyl group may be a natural or synthetic, branched or unbranched, cyclic or acyclic, substituted or unsubstituted acyl, alkyl, alkenyl and alkynyl chains of from 4 to 30 carbon atoms, such as, e.g., from 6 to 24 carbon atoms, from 8 to 20 carbon atoms or from 12 to 20 carbon atoms.

In one embodiment of the invention the cationic substance may be an acyl amine, such as, e.g. a lauryl amine (C12), palmityl amine (C16), palmitoleyl amine (C16:1), oleyl amine (C18:1) and linoleyl amine (C18:2).

In a further embodiment of the invention the cationic substance may be a mixture of oleyl amine and lauryl amine. The w/w ratio of oleyl amine to lauryl amine may be from about 0.1 to about 10, such as, e.g., from about 0.25 to about 9, from about 0.5 to about 8, from about 0.75 to about 7, from about 1 to about 6, from about 1 to about 5, from about 1 to about 4, from about 1 to about 3, from about 1 to about 2 and from about 1 to about 1.

The adjuvant according to the invention may further comprise a monoglyceride having the formula 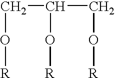
wherein R is selected from H and an acyl group containing from 6 to 30 carbon atoms with the proviso that two of the R groups are H. In a monoglyceride the acyl chains are normally placed on carbon atom 1 or 3 of the glycerol backbone, but there will often be a acyl migration between the carbon atoms 1 and 3, and the center carbon atom 2, resulting in that approximately 90% of the acyl chains will be positioned on the carbon atom 1 or 3, and about 10% will be positioned on the center carbon atom.

In the present invention is used distilled 1-monoglyceride from Danisco Ingredients (Denmark) with a purity of at least 80% w/w, such as, e.g., at least 90% w/w or at least 95% w/w. The content of diglyceride in the 1-monoglyceride is at the most 3% and the content of triglycerides and fatty acids are less than 1%.

In one embodiment of the invention, the adjuvant may be a mixture of a monoglycerides and an acyl amine, such as, e.g., mono-olein and oleyl amine.

The w/w ratio of mono-olein to oleyl amine may be from about 0.1 to about 10, such as, e.g., from about 0.25 to about 9, from about 0.5 to about 8, from about 0.5 to about 7, from about 0.5 to about 6, from about 1 to about 5, from about 1 to about 4, from about 1 to about 3, from about 1 to about 2 and from about 1 to about 1.

In a specific embodiment, the w/w ratio of mono-olein to oleyl amine may be 0.45.

The adjuvant according to the invention contains the adjuvant components, i.e. the cationic substances and optionally one or more monoglycerides in a concentration that elicits an immune response in a human or animal to an antigen administered to the human or animal.

One embodiment of the invention relates to an adjuvant dispersed in a medium. When the adjuvant is dispersed in a medium the term “adjuvant-containing medium” is used to describe the medium.

In an aspect of the invention, the medium may be in the form of an aqueous medium such as an aqueous suspension.

The medium may further comprise an immune stimulating agent such as, e.g., a CpG motif or an immune stimulating small nucleotide sequence.

As mentioned above, the adjuvant may comprise one or more cationic substances alone or together with one or more monoglycerides. The total concentration of cationic substances, either alone or if relevant together with one or more monoglycerides in an adjuvant-containing medium is at the most 25% w/v, such as, e.g., at the most 20% w/v, at the most 15% w/v, at the most 10% w/v, at the most 5% w/v, at the most 4% w/v, at the most 3% w/v, at the most 2% w/v or at the most 1% w/v.

A specific embodiment of the invention relates to an adjuvant comprising one or more cationic substances and no monoglycerides, and wherein the total amount of cationic substances in an adjuvant-containing medium is from about 0.1% w/v to about 15% w/v, such as, e.g., from about 0.25% w/v to about 12.5% w/v, from about 0.5% w/v to about 10% w/v, from about 1% w/v to about 7.5% w/v, from about 1% w/v to about 5% w/v, from about 1% w/v to about 4% w/v, from about 1% w/v to about 3% w/v, from about 1% w/v to about 2% w/v or from about 0.5% w/v to about 4% w/v.

The invention also relates to an adjuvant comprising one or more monoglycerides together with one or more cationic substances, wherein the total amount of cationic substances in an adjuvant-containing medium is from bout 0.1% w/v to about 10% w/v, such as, e.g., from about from about 0.25% w/v to about 9% w/v, from about 0.5% w/v to about 8% w/v, from about 1% w/v to about 7% w/v, from about 1% w/v to about 6% w/v, from about 1% w/v to about 5% w/v, from about 1% w/v to about 4% w/v, from about 1% w/v to about 3% w/v, from about 1% w/v to about 2% w/v or from about 0.5% w/v to about 4% w/v.

In one aspect of the invention the medium may further comprise a surface-active agent, which may be hydrophilic and inert and biocompatible, such as, e.g., Pluronic F68.

The medium may further comprise one or more physiologically acceptable additives, such as, e.g., buffering agents, such as, e.g. Tris, stabilizing agents, osmotically active agents, preservatives and pH adjusting agents.

The pH of the medium should be within the physiologically acceptable range, such as from about pH 6 to pH 8. The exact value will be determined based on the pKₐ value of the cationic substances.

An adjuvant according to the present invention can be used for the preparation of a vaccine. Such a vaccine comprises the adjuvant together with an immunogenic quantity of an antigen component and, optionally dispersed in a medium such as an aqueous medium. The vaccine composition may also comprise additional adjuvants, such as, e.g. a monoglyceride as described above.

A specific embodiment of the invention relates to a vaccine comprising one or more cationic substances and no monoglycerides, and wherein the total amount of cationic substances is from about 0.1% w/v to about 15% w/v, such as, e.g., from about 0.25% w/v to about 12.5% w/v, from about 0.5% w/v to about 10% w/v, from about 1% w/v to about 7.5% w/v, from about 1% w/v to about 5% w/v, from about 1% w/v to about 4% w/v, from about 1% w/v to about 3% w/v or from about 1% w/v to about 2% w/v.

The invention also relates to a vaccine comprising one or more monoglycerides together with one or more cationic substances, wherein the total amount of cationic substances is from bout 0.1% w/v to about 10% w/v, such as, e.g., from about from about 0.25% w/v to about 9% w/v, from about 0.5% w/v to about 8% w/v, from about 1% w/v to about 7% w/v, from about 1% w/v to about 6% w/v, from about 1% w/v to about 5% w/v, from about 1% w/v to about 4% w/v, from about 1% w/v to about 3% w/v or from about 1% w/v to about 2% w/v.

For specific non-limiting examples of the composition and preparation of adjuvant and vaccine formulations, please see the enclosed examples.

The antigen component may be selected from the group consisting of antigens from pathogenic and non-pathogenic bacteria, viruses, parasites and tumor cells, or it may be a polynucleotide.

The vaccine formulations comprising an adjuvant according to the invention may be suitable for protection or treatment of animals against a variety of disease states such as, for example, viral, bacterial or parasitic infections, cancer, allergies and autoimmune disorders. Some specific examples of disorders or disease states, which can be protected against or treated by using the methods or compositions according to the present invention, are viral infections caused by hepatitis viruses A, B, C, D & E3 HIV, herpes viruses 1, 2, 6 & 7, cytomegalovirus, varicella zoster, papilloma virus, Epstein Barr virus, influenza viruses, para-influenza viruses, adenoviruses, bunya viruses (e.g. hanta virus), coxsakie viruses, picoma viruses, rotaviruses, respiratory syncytial viruses, pox viruses, rhinoviruses, rubella virus, papovavirus, mumps virus and measles virus.

The diseases may also be bacterial infections such as infections caused by Mycobacteria causing TB and leprosy, pneumocci, aerobic gram negative bacilli, mycoplasma, staphyloccocal infections, streptococcal infections, salmonellae and chlamydiae.

The diseases may also be parasitic malaria, leishmaniasis, trypanosomiasis, toxoplasmosis, schistosomiasis, filariasis or various types of cancer such as, e.g. breast cancer, colon cancer, rectal cancer, cancer of the head and neck, renal cancer, malignant melanoma, laryngeal cancer, ovarian cancer, cervical cancer, prostate cancer.

The diseases may also be allergies due to house dust mite, pollen and other environmental allergens and autoimmune diseases such as, e.g. systemic lupus erythematosis

In some embodiments of the invention the adjuvant and/or vaccine formulation is used in a method or composition used to protect against or treat the viral disorders hepatitis B, hepatitis C, human papilloma virus, human immunodeficiency virus, or herpes simplex virus, the bacterial disease TB, cancers of the breast, colon, ovary, cervix, and prostate, or the autoimmune diseases of asthma, rheumatoid arthritis and Alzheimer's. It is to be recognized that these specific disease states have been referred to by way of example only, and are not intended to be limiting upon the scope of the present invention.

Normally, the vaccines may be administered in any convenient manner such as by parental or mucosal administration, such as, e.g. nasal, oral, rectal, vaginal, lung, aural, or skin administration, or by intramuscular, subcutaneous, intradermal or topical routes, and combinations thereof.

The nose is a very attractive route for immunization due to the fact that it is easily accessible, highly vascularized and contains a large absorptions surface. Both mucosal and systemic immune responses can be induced and immune response can be induced at distant mucosal sites, such as the vagina and rectum. Furthermore large populations can easily be immunized, with less risk of infection.

In a further aspect, the invention relates to a method of enhancing an immune response in a human or animal to an antigen administered to the human or animal, the method comprising administering an immune response enhancing effective amount of an adjuvant or a vaccine comprising the adjuvant according to the present invention to the human or animal.

### LEGENDS TO FIGURES

FIG. 1 shows responders of IFN gamma secreting cells after intranasal HIV-1 gp160/rev DNA-N3 adjuvant (0-200 μg) immunization pre- and post HIV-1 gp41 peptide booster (N3 adjuvant is an adjuvant according to the invention containing laurylamine and oleylamine). Positive results (values above the mean optical density of the negative control plus two SD) indicate that cytotoxic T-cells are formed.

FIG. 2 shows fecal IgA responses against HIV-1 gp160 envelope antigen pre- and post HIV-1 gp41 peptide booster immunization in HIV-1 gp160/rev DNA-N3 adjuvant (0-200 μg) immunized mice. Fecal wash dilution: 1:4. Positive results (values above the mean optical density of the negative control plus two SD) indicate that a mucosal response has been achieved and that the response has been effected at a distant site, such as in the GI tract.

FIG. 3 shows serum IgG response against HIV-1 gp160 envelope antigen after intranasal HIV-1 gp160/rev DNA-N3 adjuvant (0-200 μg) immunization pre and post HIV-1 gp41 peptide booster intranasally. Positive results indicate that a systemic immune response has been achieved.

FIG. 4 shows release of IL-2 in mice intranasally HIV-1 gp160/rev DNA-N3 adjuvant (0-200 μg) immunized pre and post HIV-1 gp4l peptide booster. Positive results support the formation of cytoxic T-cells.

FIG. 5 shows release of IL-1 in mice intranasally HIV-1 gp160/rev DNA-N3 adjuvant (0-200 μg) immunized pre and post HIV-1 gp4l peptide booster. Positive results support achievement of a humoral response.

The following examples are intended to illustrate the invention without limiting it in any way.

### EXAMPLES

Methods

Preparation of N3 adjuvant and vaccine composition 0.31 g mono-olein and 0.69 g oleylamine was mixed.

To obtain a 4% N3 lipid emulsion was prepared by adding to a beaker 0.4 g of the mixture of mono-olein and oleylamine, 9.6 ml 0.1 M Tris buffer, pH 8.0 and 195 μl 5M HCl. The N3 emulsion was formed by sonication for 2 minutes, whereafter the pH was adjusted to 8.0.

The final vacdne formulaton was a 1:1 mixture of the obtained N3 emulsion and a DNA solution with concentration suitable to give the final amounts of DNA used in the Examples (see Table 1).

The different doses of the N3 adjuvant used in the Examples (see Table 1) were obtained by mixing different dilutions of the 4% N3 lipid emulsion described above.

Immunization

Female 10-12 weeks old C57BI/6 mice of the H-2b haplotype (MTC, Karolinska Institute animal facility, Stockholm, Sweden) were immunized intranasally with combinations of HIV-1 rgp160BaL DNA, HIV-1 Rev/Lai DNA, gp41/MN coiled coil (aa 578-591, GIKQLQARV-LAVERY) and gp41 subtype A-D peptides (aa 661-675/MN, NEQLLELDKWASLWN, A/92UG31 aa 652-665, EKDLLALDKWANLWN, C/92BR025 aa 651-665, NEQDLL-ALDSWNLWN, D/92UG021 aa 643-657, EQELLKLDQWASLWN) and peptides and peptides representing the human 2nd CCR5 coreceptor (aa 168-182, FTRSQKEGLHYTCSSHFPYS) region (Hybaid, T-peptides, Ulm, Germany). The immunization schedule is shown in Table 1.

All DNA plasmids contain the CMV IE promoter for gene expression and the kanamycin gene and were mixed in the N3 adjuvant.

As shown in Table 1 all groups contained 7 mice. The intranasal second booster immunization was performed with a L3/peptide vaccine prepared as follows:

A 8% L3 adjuvant was prepared by combining 44 mg monoglyceride and 36 mg oleic acid in 1 ml of 0.15 M Tris buffer, pH 8.0 followed by sonication and subsequent adjustment of the pH to 8.0. Thereafter a gp4l peptide solution was admixed at a 1:1 ratio to the 8% L3 adjuvant, resulting in a 4% L3/peptide vaccine formulation with a final concentration of the peptide of 8,3 mg/ml.

Sample Collection

Blood was collected by retro-orbital bleedings, fecal pellets were from each mouse. Serum was stored at minus 20° C. until used. Fecal pellets were weighted and solubilized in Phosphate Buffered Saline (PBS) pH=7.2-7.4 (0.1 g/ml) with protease inhibitors (1 mg/ml, Sigma-Aldrich). The debris was removed by centrifugation and the supernatant stored at −70° C. When mice were sacrificed intestinal washings were collected.

Peptide Synthesis

Synthetic peptides corresponding to the gp41 neutralizing epitope (aa 661-675/ELDKWAS) (Hybaid T-peptides, Ulm, Germany) representing clade A/(92UG31), B/(MN), C/(92BR25), D/(92UG21); a gp41 clade B/(MN) peptide (Hybaid T-peptides, Ulm, Germany) located between aa 578-595 peptides representing the human and the simian CCR5 N-terminal region and 2nd loop aa 168-182, and aa 178-192 (Hybaid T-peptides, Ulm, Germany, 20, 21), a gp120 V3 loop region clade B aa 302-318 and as negative control a HIV-1 Lai rev-representing peptide aa 65-84 were synthesized by using solid phase F-moc chemistry.

The immunoreactions in the immunized mice were evaluated by the following methods.

Enzyme-linked Immunosorbent Assay for Detection of IgG and IgA Antibodies

Ninety-six well plates (NUNC-Maxisorp, Odense, Denmark) were coated with clade A-D gp4l ELDKWAS peptide, gp41 coil (QLQARVL) peptide, gp120 V3/(MN) (IHIGPGRAFV) and the human CCR5 2nd loop peptides. All peptides were solubilized in 0.1M NaHCO₃ buffer (pH 9.5 to 9.6) at coating concentration of 10 μg/ ml and added at 100 μl/well. Plates were stored overnight at room temperature and at least 24 h at 4° C.

Mouse sera were diluted in PBS (pH 7.4) with 0.5% bovine serum albumine (BSA, Boehring Mannheim, Mannheim, Germany) and 0.05% Tween 20 (Sigma, Aldrich, Sweden, AB) and 100 μl of dilutions 1:50, 1:250, 1:1250 and 1:6250 were added to each well, and incubated for 90 min at 37° C. Mucosal samples were tested as follows. Plates were blocked with 5% Blotto for 1 h at 37° C. and mucosal samples were diluted ½- 1/56 in two-fold dilutions in 2.5% Blotto and added 100 μl/well. Plates were incubated at 4° C. for 16 h and washed. Bound antibodies were detected with anti-murine IgA- and IgG specific conjugates: Horseradish peroxidase conjugated anti-mouse IgG (Biorad, Richmond), dilution 1:2000 or anti-mouse IgA (Southern Biotechnologies, Birmingham, Ala.) dilution 1:1000, was added at 100 μl/well, incubated for 2 h at 37° C., and OPD-reagent (2 mg/ml orthophenylendiamine in 0.05 M sodium citric acid pH 5.5 with 0.003% H₂O₂) was added as substrate at 100 μl/well. After a 30 minutes incubation period, the reaction was stopped by adding 100 μl/well 2.5M H₂SO₄. Absorbance was measured at 490 nm. Values above the mean optical density of the negative control plus two SD were used as the cut off and values above were considered as positive.

To determine the specific titer of antibodies directed to gp160, 96-well plates (NUNC-Maxisorp, Odense, Denmark) were coated with a recombinant gp160. This protein was solubilized in 0.1 M NaCO₃ buffer (pH 9.5 to 9.6) at coating concentration of 1 μg/ml. Plates were stored over night at room temperature and for a minimum of 24 h at 4° C. Patient sera was diluted in ten fold dilutions starting with 1:100 in PBS (pH 7.4) with 0.5% bovine serum albumine (BSA, Boehring Mannheim, Mannheim, Germany) and 0.05% Tween 20 (Sigma, Aldrich, Sweden, AB). After washing sera were diluted 1:100, 1:1000, 1:10000 and 1:100000 and 100 μl/well were added and incubated for 90 min at 37° C. Horse peroxddase conjugate anti-human IgG (Biorad, Richmond) in dilution 1:40000 were added for 2 h at 37° C. After washing, 100 μl/well OPD (2 mg/ml orthophenylendiamine in 0.01 M sodium citric/citric acid pH 5.5 with 0.03% H₂O₂) was added as substrate and the reaction mixture was incubated for 30 min. Reaction was stopped by adding 2.5M H₂SO₄. The absorbance was measured at 490 nm. Values above the mean optical density of the negative control plus 2 SD were considered as positive.

The positive control used was a human HIV-IgG pool collected from HIV-1 infected Ugandan patients, the Kabi 62 serum, monoclonal antibodies against the gp41 ELDKWAS epitope Mab 2F5 (donated by Dr. H. Katinger), monoclonal anti-gp120 V3 antibody F58/H3 and monoclonal antibodies against the 2nd CCR5 external loop Mab 2D7 (Coulter Pharmaceuticals, Palo Alto, Calif.). As negative controls pre-immunizabon serum, pre-immunization vaginal washings and fecal pellets were used. Quantificaton of IgA and IgG was performed by capture ELISA with commercially available IgA (1 mg/ml) and IgG (2 mg/ml) standards (Sigma). Values above the mean optical density of the negative control plus two SD were used as the cut off and values above were considered as positive.

IgA Purification and Quantfitaton

The fluids collected from the intestinaUfecal washings were used to isolate and analyse the IgA content. The Kaptive IgA/IgE reagents (Biotech IgG, Copenhagen, Denmark) were purchased and used as recommended by the manufacturer. Reagents for preparing an in-house murine IgA capture ELISA were purchased and a commercial murine IgA (1 mg/ml, Sigma-Aldrich) was used for preparing a standard curve. The purified IgA and the standard IgA was diluted in PBS (pH=7.2-7.4) with 5% fat-free dry-milk, 0.05% Tween 20 at ten-fold serial dilutions. One hundred micro liters per dilution was added to 96-microwellplate wells precoated with rabbit anti-murine IgA (Dakopatts AB, Sollentuna, Sweden) and incubated 1 h at 37° C. The plate wells were washed 4 times with saline with 0.05% Tween 20 before 100 μl HRP-conjugated goat anti-murine IgA was added to each well. After 1 h incubation at 37° C. plates were washed as previously described and the presence of bound conjugate was detected by using o-phenylene diamine in 0.05M sodium-citric acid, activated with 0.03% H₂O₂ OPD-reagent as substrate. The substrate reaction was terminated with 100 μl/well 2.5M H₂SO₄ and the absorbance was measured at OD490. The amounts of IgA in the mouse-samples were determined by comparing the OD-values of the test samples with the IgA standard.

B-ell Memory, IgG/IgA Synthesis In Vitro

1×10⁵ spleen cells were cultured in 200 μl of RPMI 1640 medium (Life Technologies, Scotland, UK) supplemented with 5% inactivated fetal calf serum at 37° C. in 96-flat bottomed cell culture plates (NUNC) with rgp160 (1 μg/ml) or with peptides (10 μg/ml) for 72 hrs. After washing, antigen-specific immunoglobulins were measured by ELISA as mentioned above. A positive reactivity was considered if the ELISA substrate absorbance was higher than the mean OD+2SD of the negative control mice.

Lymph nodes were collected from sacrificed mice. The lymph nodes cells were pooled for each group of mice due to low total numbers of available cells from each individual mouse. 1×10⁵ lymph node cells were cultured in 200 μl of RPMI 1640 supplemented with 5% inactivated fetal calf serum at 37° C. in 96-flat bottomed cell culture plates with rgp160 (1 μg/ml) or with peptides (10 μg/ml) for 72 hrs. After washing, antigen-specific immunoglobulins were measured by ELISA as mentioned above. A positive reactivity was considered if the ELISA substrate absorbance was higher than the mean OD+2SD of the negative control mice.

N3/L3 adjuvant for HIV-1 gp160/rev DNA and gp41 peptide booster immunization

Immunization Schedule

3 days prior to the start of the study: Pre-bleed of mice.

WEEK 0. Primary immunization, each group contains 7 mice and each was immunized intranasally with 10 μl of the vaccine formulations. By using only 10 μl of the vaccine formulation it is ensured that the vaccine formulation is only applied to the nose of the mice, and not to the mouth and lungs.

TABLE 1

| Group | μg of gp160-Rev | Dose N3 (μg)* |
|---|---|---|
| 1 | 8 | 200 |
| 2 | 8 | 100 |
| 3 | 8 | 50 |
| 4 | 8 | 20 |
| 5 | 8 | 0 |
| 6 | 0.8 | 100 |
| 7 | 0.8 | 0 |

*total amount of mono-olein and oleyl amine

WEEK 4: Blood sampling from mice for immunoanalyses.

WEEK 7: Booster-immunization with 10 μg gp41 peptide in 4% L3-adjuvant intranasally (6 μl/nostril) to all mice in groups 1-6.
Mice in group 7 received the gp41 peptides diluted in saline (10 μg/mouse, 6 μl /nostril).

WEEK 10: Blood sampling and immunoassays.

WEEK 12: Mice sacrificed. Blood and spleen cell analyses.

Results

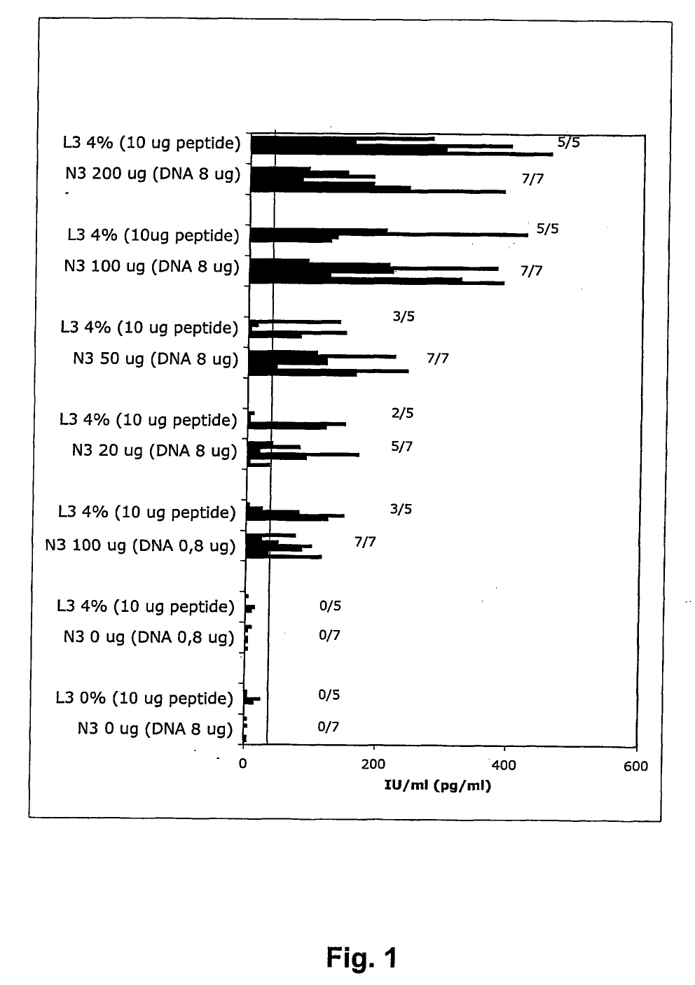

The results from the study are shown in FIGS. 1-5.

The results are seen as bars with two sets of data for each group of mice. The first (lower datagroup) shows the results obtained 4 weeks after the DNA administration and the second shows the immune response three weeks after the booster vaccination. All results indicate that unless the DNA is co-administered with the N3 adjuvant, no immune response above cut-off level can be detected in any of the groups when only 10 μl vaccine formulation is used for the nasal administration. Also shown is the dose-dependent response of the N3 adjuvant according to the present invention.

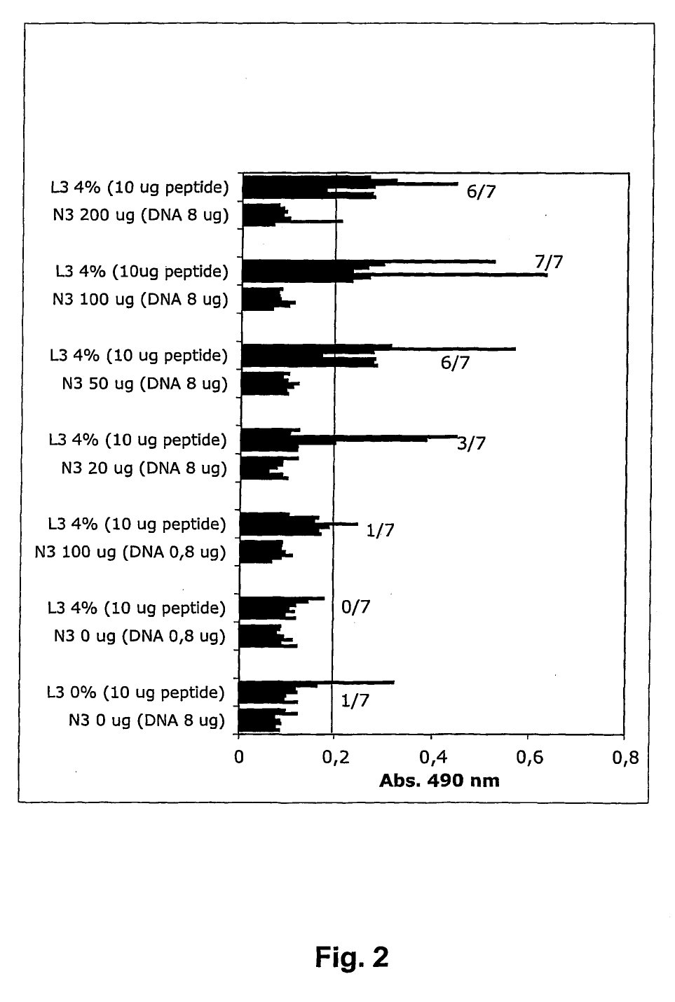

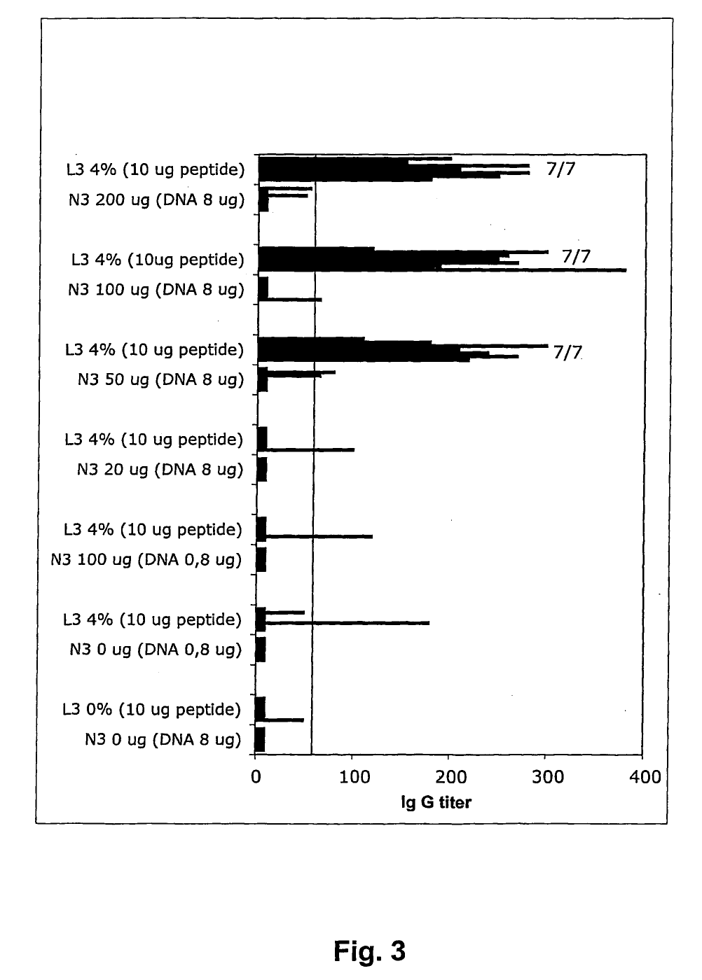

With respect to the immune responses obtained after the first immunization with HIV-1 gp160 DNA alone, no or only few or weak antibody responses were seen in serum (FIG. 3) and intestinal samples (FIG. 2). No difference was seen in animals receiving DNA with or without the N3 adjuvant.

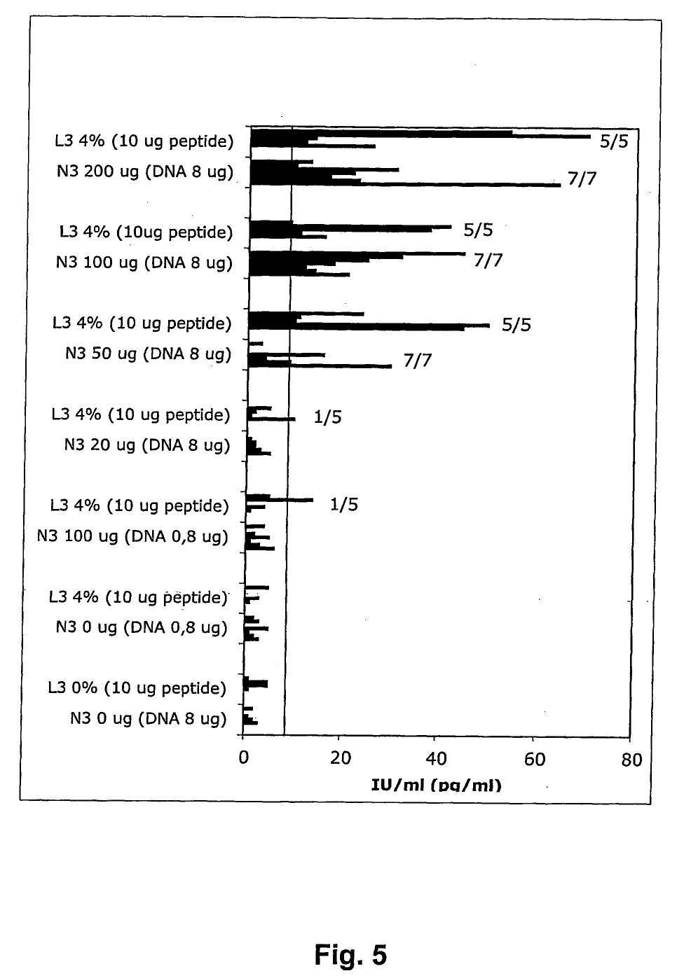

As for the cell-mediated immunity (FIG. 1) almost all (6/7 -7/7) animals receiving 8 μg DNA with 50 μg of N3 or more responded by developing IFN-gamma releasing T-cells suggesting that a cytotoxic T cell response was evoked against the HIV-1 envelope antigen. In animals receiving lower N3 adjuvant concentrations or lower gp160-DNA amounts than 8 μg, between 2-4/7 animals responded by INF-gamma responses. No HIV-1 envelope specific responses were seen in animals receiving DNA without the N3 adjuvant. Another marker for cell-mediated immunity is IL-2. As can be seen in FIG. 5, the same pattern is seen as for IFN-gamma, further strengthening the suggestion that a cytotoxic T cell response has been evoked.

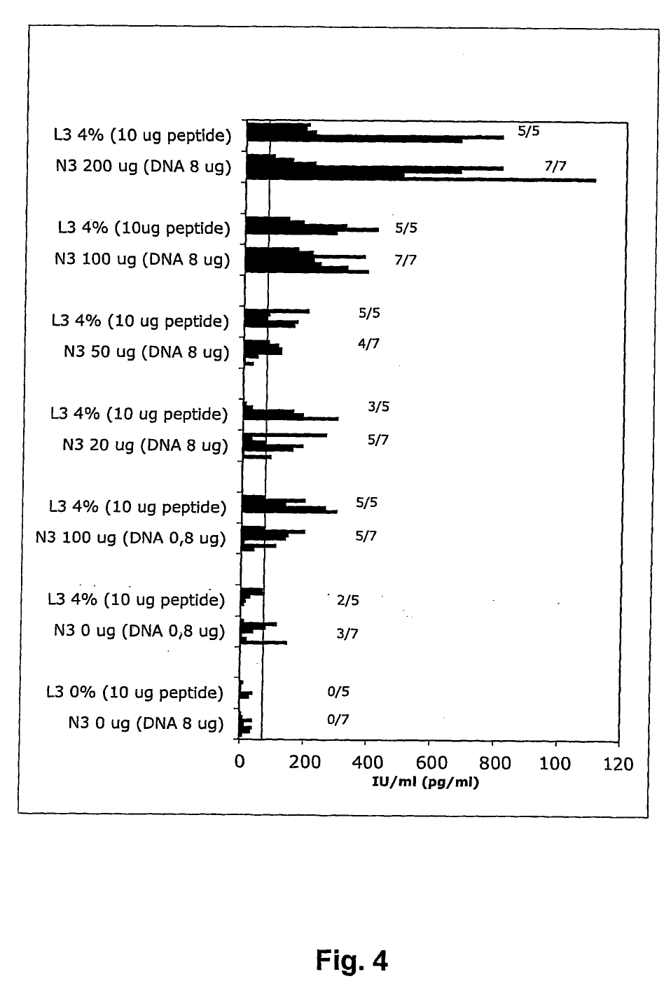

With respect to the immune responses after the HIV-1 gp41/L3 peptide booster immunization (FIG. 2 and 3), practically all animals immunized with 8 μg gp160-DNA/N3 followed by gp4l peptide in 4% L3 adjuvant developed serum IgG and intestinal IgA specific for HIV-1 envelope antigen and the HIV-1 gp41 transmembrane protein. Poor antibody responses were seen in animals receiving gp41 peptides without the L3 adjuvant (1/7 developed low amounts of antibodies to HIV-1 envelope). Another marker for a humoral immune response is IL-4. As can be seen in FIG. 4, a response of IL4 can be seen, further strengthening the suggestion that a humoral immune response has been evoked.

As for the cell-mediated immunity all animals receiving 8 μg DNA with 100 μg of N3 or more and gp41 peptide in 4% L3 adjuvant responded by developing IFN-gamma releasing T-cells suggesting that a cytotoxic T cell response was evoked against the HIV-1 envelope antigen. In animals receiving lower N3 adjuvant concentrations or lower gp160-DNA amounts than 8 μg, between 3-4/5 animals responded by INF-gamma responses. Animals immunized without the N3 adjuvant did not respond by developing detectable cell-mediated immunity.

Furthermore, when DNA was administered together with L3 instead of the N3 adjuvant, the cell-mediated immunity induced were the same as for DNA without adjuvant (in distilled water or saline), indicating that DNA and L3 is not an immune enhancing combination.

Conclusions

A clear N3-adjuvant dose-dependent enhancing effect both on antibody reactivity in serum and on mucosal surfaces was seen. At least a dose of 50 μg N3 adjuvant emulsified in a medium was needed to obtain a high frequency of IgG, IgA and IL-4 immune response.

A clear N3-adjuvant dose-dependent effect was also seen on the cell-mediated immune responses towards the HIV-1 envelope antigen. The highest amounts of INF-gamma and IL-2 cytokine responses were seen in animals obtaining a dose of 100 μg adjuvant or more, suggesting that these animals developed higher amounts of cytotoxic T cells against the HIV-1 envelope.

1. An adjuvant for use in a vaccine, the adjuvant comprising one or more cationic substances such as, e.g., acyl amines comprising from 4 to 30 carbon atoms, quaternary ammonium compounds derived from acyl amines, cationic acyl amides, amino acids conjugated to an acyl group, etc., and mixtures thereof.

2. An adjuvant according to claim 1, which—as a further adjuvant component—comprises a monoglyceride with a purity of at least 80% w/w, the monoglyceride having the formula 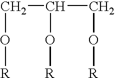
wherein R is selected from H and an acyl group containing from 6 to 30 carbon atoms with the proviso that two of the R groups are H.

3. An adjuvant according to claim 1, wherein the adjuvant components, i.e. the cationic substances and optionally monoglycerides are present in a concentration that elicits an immune response when administered to an animal.

4. An adjuvant according to claim 1, wherein the cationic substance is an acyl amine, which is saturated and/or unsaturated.

5. An adjuvant according to claim 1, wherein the cationic acyl amines is selected from the group consisting of lauryl amine (C12), palmityl amine (C16), palmitoleyl amine (C16:1), oleyl amine (C18:1) and linoleyl amine (C18:2).

6. An adjuvant according to claim 5, comprising a mixture of oleyl amine and lauryl amine.

7. An adjuvant according to claim 1, further comprising a medium.

8. An adjuvant according to claim 7, wherein the medium is aqueous.

9. An adjuvant according to claim 7, wherein the medium comprises an immune stimulating substances, such as, e.g., a CpG motif.

10. An adjuvant according to claim 7, wherein the medium further comprises a surface-active agent.

11. An adjuvant according to claim 10, wherein the surface-active agent is hydrophilic and is inert and biocompatible, such as, e.g., Pluronic F68.

12. An adjuvant according to claim 7, wherein the medium further comprises one or more physiologically acceptable additives, such as, e.g., buffering agents, stabilising agents, osmotically active agents, preservatives and pH adjusting agents.

13. An adjuvant according to claim 8, wherein the total concentration of cationic substances, either alone or if relevant together with monoglycerides and/or fatty acids, in the medium is at the most 25% w/v, such as, e.g., at the most 20% w/v, at the most 15% w/v, at the most 10% w/v, at the most 5% w/v, at the most 4% w/v, or at the most 3% w/v.

14. A vaccine comprising an adjuvant according to claim 1 together with an immunogenic quantity of an antigen component.

15. A vaccine composition according to claim 14, wherein the antigen component is selected from the group consisting of antigens from pathogenic and non-pathogenic bacteria, viruses, parasites and tumor cells.

16. A vaccine composition according to claim 15, wherein the antigen component is a polynucleotide.

17. A vaccine composition according to claim 14, further containing an aqueous medium.

18. A vaccine composition according to claim 14, wherein the adjuvant comprises a mixture of mono-olein and oleyl amine.

19. A vaccine composition according to claim 18, wherein the w/w ratio of mono-olein and oleyl amine is about 0.45.

20. A vaccine composition according to claim 18, wherein the total amount of mono-olein and oleyl amine is at least 40 μg, such as, e.g., at least 50 μg, at least 55 μg, at least 60 μg, at least 70 μg, at least 80 μg, at least 90 μg or at least 100 μg.

21. A vaccine composition according to claim 14, wherein the composition comprises additional adjuvants.

22. A vaccine composition according to claim 13, wherein the composition is in a form suitable for parenteral or mucosal administration.

23. A vaccine composition according to claim 22, wherein the composition is in a form suitable for administration to the mucosa of the nose, mouth, vagina, rectum or intestine.

24. A method of enhancing an immune response in a human or animal to an antigen administered to said human or animal, the method comprising administering an immune response enhancing effective amount of an adjuvant according to claim 1 to the human or animal.

25. A method of immunizing a human or an animal, the method comprising administering a vaccine composition according to claim 14.
